(19) United States
(12) Patent Application Publication
(10) Pub. No.: US 2020/0352849 A1
(43) Pub. Date: Nov. 12, 2020

(54) Topical Skin Formulations and Wound Care Products with Integrated CBD Delivery Mechanisms for Skin Rejuvenation, Wound Care and Healing, Pain and Itch Relief, and Scar Prevention and Treatment

(71) Applicant: Adam M. Rotunda, Irvine, CA (US)

(72) Inventor: Adam M. Rotunda, Irvine, CA (US)

(21) Appl. No.: 16/868,492

(22) Filed: May 6, 2020

Related U.S. Application Data
(60) Provisional application No. 62/844,366, filed on May 7, 2019.

(51) Int. Cl. A61K 9/00; A61K 31/05; A61K 31/573; A61K 31/7036; A61K 31/635; A61K 8/67; A61K 8/73; A61K 8/86; A61Q 19/00

(52) U.S. Cl. CPC A61K 9/0014; A61K 31/05; A61K 31/573; A61K 31/7036; A61K 45/06; A61K 8/671; A61K 8/735; A61K 8/86; A61Q 19/00; A61K 31/635

(57) ABSTRACT

Cannabidiol (CBD) is one of the most abundant and physiologically active phytocannabinoids in Cannabis plants. The chemical has well-known mechanisms that have been the potential to treat and prevent numerous inflammatory, immunologic, anti-infectious, sun-damaged, age-related (wrinkling, drying, hair loss), and neurogenic-related (anti-pain, anti-itch) diseases as well as heal traumatized skin. The incorporation of CBD on or within topically applied formulations or within wound care dressings, biomaterials skin substitutes, or skin adhesives is a novel approach to alleviate, prevent and cure acute and chronic skin disease, and promote normal healing and recovery after injury.

### CROSS-REFERENCE TO RELATED APPLICATIONS

This application claims priority to U.S. application Ser. No. 62/844,366 filed May 7, 2019, the teachings of which are expressly incorporated herein by reference.

### STATEMENT RE: FEDERALLY SPONSORED RESEARCH/DEVELOPMENT

Not Applicable

### BACKGROUND

### 1. Technical Field

The present disclosure relates generally to the field of therapeutic uses of cannabinoids. More particularly, the present disclosure relates to the integration of cannabidiol delivery mechanisms into topical medical product.

### 2. Related Art

Cannabinoids are a diverse set of chemical compounds that bind to special receptors in the human body that make up the endocannabinoid system. It has been known for some time now that the human body generates endogenous substances that bind to cannabinoid receptors, such as anandamide and 2-arachidonoylglyceral (2-AG). Several endocannabinoids also bind to other receptors, such as the GPR55 receptor and vanilloid receptors. Currently, it is thought that more than 200 endocannabinoids and related substances exist which are produced by the human body and either bind to the cannabinoid receptors or otherwise complement the function of endocannabinoids.

The main exogenous source of cannabinoids are phytocannabinoids, which are found primarily in the cannabis plant, as well as in certain other plants, including E. purpurea, E. angustifolia, A. olaracea, Helicrysum, and R. marginata. In the cannabis plant, phytocannabinoids and terpenes are manufactured in resin glands (trichomes) present on the flowers and the main fan leaves of late-stage cannabis plants.

Over 100 phytocannabinoids have been identified as deriving from the cannabis plant. The most studied phytocannabinoids are tetrahydrocannabinol (THC) and cannabidiol (CBD).

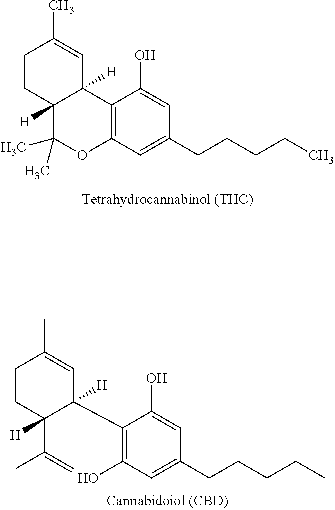

THC is generally considered to be the primary psychoactive phytocannabinoid derived from the cannabis plant. In the cannabis plant, THC is produced via the decarboxylation of its precursor tetrahydrocannabinolic acid (THCA), with the decarboxylation conversion process being accelerated via the drying of the cannabis plant, and rapidly accelerated when THCA is heated or burned.

CBD is generally considered to be a non-psychoactive phytocannabinoid derived from the cannabis plant. Similar to THC, CBD is produced via decarboxylation from its precursor cannabidiolic acid (CBDA) in a similar fashion. However, CBD is considered to have little binding affinity for either of the two confirmed cannabinoid receptors, CB₁ and CB₂. Rather, CBD modulates several non-cannabinoid receptors and ion channels, as well as acting through various receptor-independent pathways, such as by delaying the reuptake of endogenous neurotransmitters (such as anandamide and adenosine) and by enhancing or inhibiting the binding action of certain G-protein coupled receptors.

One interaction of CBD that has been discovered is that it serves as a partial agonist of the 5-HT_{1A} (hydroxytryptamine) serotonin receptor, which is a G protein-coupled receptor that mediates inhibitory neurotransmission, resulting in relief of anxiety and depression in humans. Multiple anxiolytic and antidepressant partial or full 5-HT_{1A} receptor agonists, such as buspirone and tandospirone, are presently in common medical use.

Another interaction of CBD is with the transient receptor potential cation channel subfamily V member 1 (TrpV1) receptor, also known as the capsaicin receptor and the vanilloid receptor 1. The TrpV1 receptor mediates pain and temperature perception. Currently, some products are in clinical use which rely on TrpV1 receptor agonists, such as capsaicin and resiniferatoxin, for the alleviation of localized pain via prolonged application resulting in long term desensitization of those receptors. For example, 8% capsaicin patches which rely on this interaction have recently entered into clinical use, with novel preparations containing higher amounts of capsaicin under clinical trials. Thus, CBD may also provide alleviation of medical conditions via these interactions as well.

Recent studies have also indicated that CBD may function as an antagonist for the G protein-coupled receptor 55 (GPR55). GPR55 is widely expressed in the brain, especially in the cerebellum, but it is currently considered to be an “orphan receptor,” as its physiological function remains unclear and there remains certain uncertainty as to whether it belongs to a larger family of receptors, such as the cannabinoid receptors, and should be considered to be the “CB₃” receptor. Some research has indicated that GPR55 receptor activation increases the level of intracellular calcium and inhibits M current. There also is a growing body of evidence that GPR55 may play a role in controlling cell proliferation, and thus may be implicated as a biomarker or target in certain cancer therapies. According to some sources, THC operates as an agonist for the GPR55 receptor, causing its activation, while CBD is one of its few known antagonists, preventing its activation. Accordingly, administration of CBD therapeutics may have a substantial effect with regard to the conditions or other therapeutics activating or affected by activation of the GPR55 receptor.

These are only some of the known or suspected biological interactions of CBD. Thus, CBD may be seen to have many potential uses as a as a therapeutic agent for numerous pathological conditions. Known pharmacologic effects of CBD include anti-oxidation, anti-inflammation, anti-bacterial, analgesia (anti-pain), neuroprotection, immunomodulation, ant-psychotic, ant-fibrosis, anti-coagulation, anti-nausea, ant-fatigue and muscle tension, anti-depression, and anti-convulsant activity. These effects have current or potential applications in: Alzheimer's disease, Parkinson's disease, multiple sclerosis, epilepsy, Huntington's disease, pain, hypoxia-ischemia injuries (i.e., cardiovascular and nerve disease), cancer, anxiety, depression, hypertension, nausea, inflammatory diseases (i.e., bowel disease, arthritis, cardiovascular disease, autoimmune diseases), and complications from diabetes.

The skin is the largest organ of the human body. As with any organ, it is prone to injury, disease, loss of function, and because it is visually accessible, changes in appearance. Skin injury and changes in appearance and function may result from iatrogenic causes (i.e., medically-induced reasons such as surgery, radiation and medication side effects); accident (trauma, burns, falls, sharp and blunt injury, abrasions and lacerations); aging and age-related changes in hormones (senescence or ‘wear and tear’); as well as changes from acute and chronic sun exposure (sunburns, wrinkling, thinning, sun induced drying and changes in color).

Superficial injury and loss of function of the skin includes skin pain, scarring, redness and irritation (dermatitis); hair loss (due to age, genetics or hormones); changes in color (darkening and lightening); infection (bacterial, fungal or viral); skin thickening and thinning; and prolonged, poor or delayed healing. Deep skin injury may lead to hypertrophic and keloid scars, hair loss, distortion of shape, and loss of function (such as loss or increased sweating).

Systemic disease such as diabetes, autoimmune disease (such as lupus), immunosuppression (i.e., due to cancer, or drugs like chemotherapy, steroids and other immunosuppressive drugs, or HIV and other viral illnesses or infection), as well as local skin disease and injury (such as infections, dermatitis, poor blood supply or circulation, and burns) contribute to prolonged wound healing and loss of function.

Numerous creams, ointments and other topical products, as well as devices and wound care products are used by physicians and/or are purchased by patients over the counter to facilitate wound healing, minimize scarring, counter infection, minimize pain, reduce signs of ageing and promote hair growth. Some examples include topical, aqueous as well as non-aqueous (i.e., oil or ointment based) creams, emollients, sprays, tinctures, gels, lubricants, and balms.

Specific examples include agents used as anti-inflammatories (i.e., hydrocortisone, aloe vera, topical vitamins and minerals) for skin rashes; anti-bacterial products (antibiotics like neomycin, polymyxin B, bacitracin zinc, and polymyxin B sulfate, and silver sulfadiazine), antifungals (nystatin and -azole containing products), and antivirals; skin rejuvenation products (skin moisturizers and hydration creams, vitamin A derivatives, hyaluronic acids, glycerol, polyethylene glycol and dimethicone containing products); analgesics (i.e., benzocaine, lidocaine, camphor, methyl salicylate and menthol, and essential oils, steroids, vitamins, herbs, MSM, glucosamine, and capsaicin); anti-itch products that can be used for allergic reactions, rashes, dermatitis, psoriasis (i.e., camphor, menthol, phenol, pramoxine and diphenhydramine, or topical anesthetics); sunscreens and sunburn relief products (i.e., aloe vera, steroids, and oatmeal containing products); lip balms (containing beeswax or carnauba wax, camphor, cetyl alcohol, lanolin, paraffin, and petrolatum); topical products for wound healing (including honey, petrolatum, iodine, mineral oil); shampoos for dandruff (containing selenium, zinc, coal, salicylic, ketoconazole); hair growth products (containing minoxidil, finasteride, biotin and other vitamins and minerals, and essential oils, for example); and scarring treatment and prevention product and devices (such as vitamins, silicone dioxide and polysiloxanes sheets, cyanoacrylates, retinoic acid and retinol derivatives, as well as onion derivatives). Furthermore, over-the-counter bandages used for wound healing include non-stick fabric and as well as synthetic and polyethylene gauzes, occlusive and non-occlusive bandages, skin adhesives (cyanoacrylates), hydrocolloid and other polymer-based dressings. Other wound care materials are prescription based and may be compromised of epidermal (keratinocyte) and dermal (collagen) skin components, as well as skin growth factors meant to facilitate skin growth, repair, and wound healing.

Because investigation into the therapeutic potential of CBD is ongoing, there is likewise a need in the art for novel ways to synergistically integrate the therapeutic capabilities of CBD with topical products to form novel topical medical systems.

### BRIEF SUMMARY

To solve these and other problems, according to various exemplary embodiments, an augmented topical medical system is contemplated as comprising an topical product configured to perform at least a first medical function ensuing from the application of the topical product at a site of application, and a drug release mechanism in physical association with the topical product, with the drug release mechanism being configured to deliver cannabidiol at the site of application, and with the delivery of cannabidiol at the site of application being configured to augment the performance of the first medical function by the topical product.

According to various of these exemplary embodiments, the augmented topical medical system may comprise one or more of a cream, an ointment, an emollient, a spray, a tincture, a gel, an anti-inflammatory product, an anti-fungal product, an anti-viral product, a skin rejuvenation product, a moisturizing product, an analgesic product, an anti-itch product, a sunscreen product, a sunburn relief product, a lip balm, a transdermal patch, a wound healing product, an anti-dandruff product, a hair-growth promoting product, a scar treatment and prevention product, a wound care product.

Where the topical product comprises an anti-inflammatory product, the topical product may comprise, for example, one or more of: hydrocortisone, aloe vera, topical vitamins and minerals.

Where the topical product comprises an anti-bacterial product, the topical product may comprise, for example, one or more of: neomycin, polymyxin B, bacitracin zinc, polymyxin B sulfate, silver sulfa.

Where the topical product comprises a skin rejuvenation product, the topical product may comprise, for example, one or more of: a moisturizer, a hydration cream, a vitamin A derivative, a hyaluronic acid, glycerol, polyethylene glycol, dimethicone.

Where the topical product comprises an analgesic product, the topical product may comprise, for example, one or more of: benzocaine, lidocaine, camphor, methyl salicylate, a steroid, glucosamine, methylsulfonylmethane, capsaicin.

Where the topical product comprises an anti-itch product, the topical product may comprise, for example, one or more of: camphor, menthol, phenol.

Where the topical product comprises a wound care product, the topical product may comprise, for example, one or more of: honey, petrolatum, iodine, mineral oil, an occlusive bandage, a non-occlusive bandage, a synthetic gauze, a polyethylene gauze, a hydrocolloid dressing, a cyanoacrylate adhesive, a skin growth factor, a keratinocyte, collagen.

Where the topical product comprises a scarring treatment and prevention product, the topical product may comprise, for example, one or more of: silicone dioxide, a polysiloxane sheet, a cyanoacrylate, retinoic acid, a retinol derivative, an onion derivative.

Where the topical product comprises a lip balm, the topical product may comprise, for example, one or more of: beeswax, carnauba wax, camphor, acetyl alcohol, lanolin, paraffin, petrolatum.

Where the topical product comprises an anti-dandruff product, the topical product may comprise, for example, one or more of: selenium, zinc, coal tar, salicylic, ketoconazole.

It in contemplated that in certain embodiments, the drug release mechanism configured to deliver cannabidiol at the site of application may comprise integration of cannabidiol into the formulation of a topical product. In other embodiments, the drug release mechanism configured to deliver configured to deliver cannabidiol at the site of application may comprise at least partial permeation of cannabidiol into the material of a topical product.

According to further embodiments, the herein the drug release mechanism configured to deliver configured to deliver cannabidiol at the site of application may comprise a reservoir, the reservoir being operative to elute cannabidiol at the site of application. The reservoir may be associated with a controller operative to actuate release of cannabidiol from the reservoir. The reservoir may be operative to actuate release of cannabidiol from the reservoir based upon one or more of: a predetermined timing, receipt of a release command, detection of a physiological condition. The controller may comprise a microchip. The reservoir may comprise, for example, one or more of: an aqueous formulation; a non-aqueous formulation; a polymer formulation, a liposomal formulation, a micro-encapsulation formulation, an ion-exchange resin formulation, a hydrogel formulation, or combinations thereof.

The drug release mechanism configured to deliver cannabidiol at the site of application may also comprise an adherent coating configured to elute cannabidiol, and the physical association with the topical product may comprise the adherent coating being adhered to at least a portion of a surface of the topical product.

### DETAILED DESCRIPTION

According to various embodiments of the present disclosure, augmented topical medical systems are are contemplated in which a topical product configured to perform at least a first medical function is augmented by the physical association therewith of a drug release mechanism configured to delivery cannabidiol (CBD) at the site of administration, thereby augmenting the performance of the first medical function. As described above, CBD delivery at the site of administration may be beneficial in treating or aiding in the treatment of a large number of conditions, through administration via a variety of biochemical pathways or combinations of biochemical pathways. Thus, it may be seen that the therapeutic application of CBD for augmentation of the performance of the first medical function of the topical product may be customized in a variety of ways particular to, among other things, the type of topical, the medical function of the topical product to be augmented, the condition or conditions being treated by the administration of the topical product, and the particular needs of the recipient of the topical product, which is primarily contemplated to be a human patient but may also be other organism which may benefit from administration of the presently contemplated augmented topical medical systems.

The following disclosure exemplifies a number of ways in which such contemplated augmented topical medical systems containing a drug release mechanism for the delivery of CBD at the site of administration may be customized, but it is to be understood that the ways in which such customization may occur is essentially infinite, and as such the foregoing discussed contemplated augmented topical medical systems are to be understood as exemplary and illustrative, and that the scope of the presently contemplated disclosure is not to be merely limited to the particular embodiments discussed herein, but rather as including the full scope of all varieties, combinations, and potential combinations of such customized augmented topical medical systems.

According to one exemplary embodiment of an augmented topical medical system having an topical product with a drug release mechanism being in physical association with the topical product and being configured to deliver cannabidiol at the site of application comprises an a cream, ointment, lotion, oil, emulsion, shampoo, or spray for application directly to the skin, with cannabidoil being directly emulsified or dissolved within the topical product. Following application of the topical product, the CBD may dissociate from or otherwise migrate from the applied topical product and to the site of application, which may occur via a number of processes, depending on the configuration of the topical product and the drug delivery mechanism. For example, it may be seen that in the case where the topical product is configured to be bioabsorbed by the skin, the CBD associated therewith may likewise be bioabsorbed along with or alongside the topical product. It may also be seen that in cases where the topical product is not configured to be bioabsorbed, but rather is to reside at the point of application and subsequently be removed from the point of application, the CBD may be configured remain with the patient and may be bioabsorbed.

It may also be seen that in other embodiments of an augmented topical medical system, the CBD may be confined within a distinct drug release mechanism that it's not itself embodied by formulation of the topical product, the CBD may be confined or otherwise configured to remain in place in physical association with the topical product via the drug release mechanism, with the CBD eluting or otherwise being administered from the drug release mechanism, such as in release or elution of CBD from a reservoir, depot, coating, a permeated material, or other method of confining CBD prior to delivery.

It may be seen that in the case that the drug release mechanism is a reservoir, depot, coating, permeated material, or other method of physical confining CBD prior to release or elution to the area of administration, the drug release mechanism may be any type of reservoir, depot, coating, permeated material known to be able to confine and subsequently allow release of small molecules such as CBD. As used in this disclosure, the term “reservoir” is to be understood as any method known or developed in the future that is capable of at least temporarily confining or containing small molecules such as with the CBD, and shall be inclusive of confinement techniques referred to under various names such as reservoirs, depots, permeated materials, permeated coatings, dissociative time release capsules, etc. In such reservoirs, the CBD may be contained by itself or in solution with or otherwise in association with other compounds, such as carrier oils or other carrier materials. Many drug reservoirs are known in the art which may serve to allow long-term sustained release of small molecules, or on-demand delivery. Some types of reservoirs may be configured deliver their supply passively over time, as in, for example, polymer formulations, liposomal formulations, micro-encapsulation formulations, ion exchange resins, hydrogels, or combinations of these. In other cases, reservoirs may be used in conjunction with microelectromechanical systems, such as microfluidic systems, in order to enable delivery from the reservoir in other ways (an actively driven reservoir). Other types of reservoirs may be configured to release CBD in response to external stimuli, such as heat, light, or the presence of other small molecules, such as, for example, exposure to blood, plasma, sweat, or any constituent components of skin, which may be seen to be useful in connection with a topical system. For example, it may be seen that a topical device such as a splint or bandage may contain associated therewith a reservoir of CBD which may be delivered following application of the splint or bandage. For example, in the case of the splint, the reservoir may be, for example, a coating on the surface of the splint meant to contact the patient's skin. To provide another example, in the case of the bandage, the reservoir may be a portion of the bandage or a pad or attachment to the bandage which may be at least partially permeated with CBD. These examples are to be understood as merely illustrative, as it may be seen that the combinations of possible systems is potentially limitless.

It may also be seen that reservoirs may be provided with an embedded fluid port or other mechanism to permit the reservoir to be refilled. Such reservoirs, rather than the drug delivery mechanism for delivery of CBD itself, may instead be for treatment of the first medical condition, with the drug delivery mechanism being associated with the reservoir. For example, a hemostatic dressing which contains a reservoir of a blood clotting agent may in addition contain a second reservoir (such as a coating) which may be configured to deliver CBD, or the reservoir of the blood clotting agent may also contain CBD. It may thus be seen that a first reservoir may itself deliver one medicament (the hemostatic agent, in the example of the hemostatic bandage) while CBD is also delivered via elution from an associated second reservoir (for example, a strip, liner, or second portion of the hemostatic bandage). These types of configurations may be useful where storage requirements for CBD and the medicament may differ in order to enhance efficacy or shelf life, such as where CBD is desired to be contained in solution with a carrier while the medicament is desired to remain a dry powdered material activated upon intermingling with a fluid, such as blood or plasma in the case of a hemostatic powder. In situations where it may not be necessary to deliver CBD and another agent from two reservoirs, the delivery action from a single reservoir may deliver both the medicament contained within the reservoir as well as CBD. For example, transdermal patches are known which may have separate drug layers, with some drugs contained within a liquid compartment or solution or suspension within the patch, while other drugs are contained within the adhesive layer of the patch. In this way, it may be seen that the delivery parameters of such as rate of delivery of the drugs contained in the different layers or compartments, or within the patch itself vs. within the adhesive component, may be controlled.

Further, it may also be seen that microfluidic devices in addition to an active or passive reservoir may be utilized as components within the present disclosures. For example, CBD may be used with an actively driven reservoir, a passive micropump, an electrostatic micropump, or an active micropump, or may otherwise be included within those components in a fashion configured to allow CBD to elute from the component following application at the site if application. Further, it may be seen that electrical components, which may or may not overlap with microfluidics devices may be utilized as well as either the topical device or to form the drug release mechanism.

It may be seen that in such cases where portions of or the entirety of a topical product of any type are configured to be bioabsorbed or to be otherwise internalized by the body, CBD may be bound up within the topical and be released at the site of administration as a consequence of such bioabsorption or internalization. It may also be seen that via modification of the way in which CBD is bound up or otherwise contained, the parameters of its release may be affected, as in a passive drug release mechanism such as a depot injection where CBD may be formulated as a component of a polymer formulation, a micro-encapsulation formulation, an ion-exchange resin formulation, a liposomal formulation, or combinations thereof.

According to other exemplary embodiments, the topical device may be other types of devices, such as a transdermal patches. Furthermore, it is contemplated that the CBD drug release mechanism may include aspects for active or passive control of delivery beyond mere time-release as a function of chemical properties of the formulation. For example, it may be seen that the drug release mechanism may be controlled by a microchip to release CBD as a function of a number of possible parameters, such as detecting a physiological condition or the receipt of a release command. For example, a system is contemplated in which a patient or doctor may, via transmission of a signal (electrical, radio, light, or otherwise), trigger the drug release mechanism to release CBD, or situations in which the system may detect (via, for example, a sensor) conditions such as elevated skin temperature or elevated heart rate, and release CBD upon detection of such conditions.

It may be seen that the delivery of CBD according to the presently described systems may aid in synergistically augmenting the performance of at least one of the medical functions of the topical product. For example, in the case of a topical product which serves an analgesic or pain-relieving function, the delivery of CBD at the site of application may synergistically aid in the analgesic function. Likewise, for a topical product for wound healing and/or prevention of scar formation, CBD delivery at the site of administration may synergistically aid in wound healing or scar formation. It is not critical that the CBD delivery at the site of the administration aids in the treatment of each and every one of the possible or actual medical functions of any given topical product, or even that CBD delivery aids in treatment of the primary medical function of a given topical product. Many topical products are recognized as delivering treatment in a number of aspects. For example, in the case of a hemostatic gauze applied for the primary purpose of stemming bleeding from a wound, the synergy flowing from the inclusion of the CBD drug delivery device may be the result of CBD's anti-anxiety effects, which may relieving anxiety in the patient caused from the wound. While application of the hemostatic gauze may be seen to directly relieve anxiety in the patient in the holistic sense (the patient is being relieved of anxiety caused by the presence of an untreated wound by receiving treatment for their wound), anxiety relief can almost never be said to the “primary” medical purpose of application of a hemostatic gauze to a wound. In this sense, it may be seen that the inclusion of the CBD drug release mechanism provides substantial synergy in treatment of a first medical function (anxiety relief) that is also performed by the application of the topical product, but that the first medical function is not necessarily the primary medical function for which the topical product has been applied. Rather, the first medical function can be a secondary medical effect of the topical product for which synergy is achieved via administration of CBD.

A similar paradigm may be seen in many other situations where the a patient may experience symptoms such as anxiety due to, for example, a condition resulting in an undesired physical appearance, such as in cases of rashes, hair loss, dandruff, sunburn, etc. In each of these cases and in others, even if administration of CBD may not itself substantially contribute to the direct mitigation of these conditions, the co-administration of CBD may serve to directly reduce anxiety, which will coordinate and synergize with the anxiety relief resulting from mitigation of those conditions. Similarly, other non-anxiety conditions relieved by CBD directly may be seen to be likewise not the “primary” purpose of the administration of the topical device, but may be a secondary or tertiary purpose or result, while still being a “first medical function” of the topical product augmented by delivery of CBD.

The above description is given by way of example, and not limitation. Given the above disclosure, one skilled in the art could devise variations that are within the scope and spirit of the invention disclosed herein, including various ways of physically associating the CBD drug release mechanism with the topical product, or of configuring the CBD drug release mechanism to release CBD. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. An augmented topical medical system, the augmented topical medical system comprising:
a topical product configured to perform at least a first medical function ensuing from application of the topical product at a site of application; and
a drug release mechanism in physical association with the topical product, the drug release mechanism being configured to deliver cannabidiol at the site of application;
wherein the delivery of cannabidiol at the site of application is configured to augment the performance of the first medical function by the topical product.

2. The augmented topical medical system of claim 1, wherein the topical product comprises one or more of: a cream, an ointment, an emollient, a spray, a tincture, a gel, an anti-inflammatory product, an anti-fungal product, an anti-viral product, a skin rejuvenation product, a moisturizing product, an analgesic product, an anti-itch product, a sunscreen product, a sunburn relief product, a lip balm, a transdermal patch, a wound healing product, an anti-dandruff product, a hair-growth promoting product, a scar treatment and prevention product, a wound care product.

3. The augmented topical medical system of claim 2, wherein the topical product is an anti-inflammatory product comprising one or more of: hydrocortisone, aloe vera, topical vitamins and minerals.

4. The augmented topical medical system of claim 2, wherein the topical product is an anti-bacterial product comprising one or more of: neomycin, polymyxin B, bacitracin zinc, polymyxin B sulfate, silver sulfa.

5. The augmented topical medical system of claim 2, wherein the topical product is an skin rejuvenation product comprising one or more of: a moisturizer, a hydration cream, a vitamin A derivative, a hyaluronic acid, glycerol, polyethylene glycol, dimethicone.

6. The augmented topical medical system of claim 2, wherein the topical product is an analgesic product comprising one or more of: benzocaine, lidocaine, camphor, methyl salicylate, a steroid, glucosamine, methylsulfonylmethane, capsaicin.

7. The augmented topical medical system of claim 2, wherein the topical product is an anti-itch product comprising one or more of: camphor, menthol, phenol.

8. The augmented topical medical system of claim 2, wherein the topical product is a wound care product comprising one or more of: honey, petrolatum, iodine, mineral oil, an occlusive bandage, a non-occlusive bandage, a synthetic gauze, a polyethylene gauze, a hydrocolloid dressing, a cyanoacrylate adhesive, a skin growth factor, a keratinocyte, collagen.

9. The augmented topical medical system of claim 2, wherein the topical product is a scarring treatment and prevention product comprising one or more of: silicone dioxide, a polysiloxane sheet, a cyanoacrylate, retinoic acid, a retinol derivative, an onion derivative.

10. The augmented topical medical system of claim 2, wherein the topical product is a lip balm comprising one or more of: beeswax, carnauba wax, camphor, acetyl alcohol, lanolin, paraffin, petrolatum.

11. The augmented topical medical system of claim 2, wherein the topical product is an anti-dandruff product comprising one or more of: selenium, zinc, coal tar, salicylic, ketoconazole.

12. The augmented topical medical system of claim 1, wherein the drug release mechanism configured to deliver configured to deliver cannabidiol at the site of application comprises integration of cannabidiol into the formulation of a topical product.

13. The augmented topical medical system of claim 1, wherein the drug release mechanism configured to deliver configured to deliver cannabidiol at the site of application comprises at least partial permeation of cannabidiol into the material of a topical product.

14. The augmented topical medical system of claim 1, wherein the drug release mechanism configured to deliver configured to deliver cannabidiol at the site of application comprises a reservoir, the reservoir being operative to elute cannabidiol at the site of application.

15. The augmented topical medical system of claim 14, wherein the reservoir is associated with a controller operative to actuate release of cannabidiol from the reservoir.

16. The augmented topical medical system of claim 15, wherein the reservoir is operative to actuate release of cannabidiol from the reservoir based upon one or more of: a predetermined timing, receipt of a release command, detection of a physiological condition.

17. The augmented topical medical system of claim 15, wherein the controller comprises a microchip.

18. The augmented topical medical system of claim 14, wherein the reservoir comprises one or more of: an aqueous formulation; a non-aqueous formulation; a polymer formulation, a liposomal formulation, a micro-encapsulation formulation, an ion-exchange resin formulation, a hydrogel formulation, or combinations thereof.

19. The augmented topical medical system of claim 1, wherein the drug release mechanism configured to deliver cannabidiol at the site of application comprises an adherent coating configured to elute cannabidiol, and wherein the physical association with the topical product comprises the adherent coating being adhered to at least a portion of a surface of the topical product.
